(19) United States
(12) Patent Application Publication
(10) Pub. No.: US 2003/0171437 A1
(43) Pub. Date: Sep. 11, 2003

(54) Pharmaceutical composition

(76) Inventors: Jurij Holinej, Hackettstown, NJ (US); Yatindra Joshi, Princeton, NJ (US); Anees A. Karnachi, Hillsborough, NJ (US); Maha Y. Khaled, Livingston, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/383,041

(22) Filed: Mar. 6, 2003

Related U.S. Application Data
(60) Provisional application No. 60/362,351, filed on Mar. 7, 2002.

(51) Int. Cl. A61K 31/195

(52) U.S. Cl. 514/567

(57) ABSTRACT

A method of treating a cyclooxygenase-2 dependent disorder or condition comprising administering a solid formulation of 5-methyl-2-(2′-chloro-6′-fluoroanilino)phenylacetic acid which has a residual moisture within predetermined levels.

This invention relates to compositions for the treatment of cyclooxygenase-2 mediated diseases and methods for stabilizing pharmaceutical compositions useful for the treatment of cyclooxygenase-2 mediated diseases.

In particular, this invention relates to compositions that comprise 5-methyl-2-(2′-chloro-6′-fluoroanilino)phenylacetic acid.

It has been surprisingly found that when the drug substance 5-methyl-2-(2′-chloro-6′-fluoroanilino)phenylacetic acid is formulated in solid form, e.g., in tablet form, the stability of the drug substance is increased by increasing the moisture content of the tablet, within a relatively narrow range, beyond which the stability decreases. That is, certain degradation products of 5-methyl-2-(2′-chloro-6′-fluoroanilino)phenylacetic acid are formed at a greater rate in dryer tablets, which is contrary to the typical behavior of drug substances. In general, moisture, in the form of water, is detrimental to drug stability, and packaging for pharmaceutical formulations frequently contains some form of desiccant material to minimize moisture levels.

This invention provides compositions for treating cyclooxygenase-2 dependent disorders or conditions comprising 5-methyl-2-(2′-chloro-6′-fluoroanilino)phenylacetic acid. The compositions comprise between about 50 and about 400 mg of 5-methyl-2-(2′-chloro-6′-fluoroanilino)phenylacetic acid and have a residual moisture level (defined herein as “loss on drying” or “LOD,” as opposed to total moisture or moisture determined according to the Karl Fischer method) between about 1.5% and about 5%. In certain embodiments, a composition comprising between about 50 and about 200 mg of 5-methyl-2-(2′-chloro-6′-fluoroanilino)phenylacetic acid will have an LOD between about 2% and 5%, or between about 2.1% and about 4.5%. In other embodiments, a composition comprising about 400 mg of 5-methyl-2-(2′-chloro-6′-fluoroanilino)phenylacetic acid will have an LOD between about 1.5% and about 4%, or between about 1.7% and about 3.5%. In certain embodiments, the compositions are tablets, and in other embodiments, film coated tablets.

In another aspect, the invention provides dried granulations useful for making pharmaceutical compositions. The dried granulations can comprise 5-methyl-2-(2′-chloro-6′-fluoroanilino)phenylacetic acid, microcrystalline cellulose, lactose monohydrate, and croscarmellose sodium, where the residual moisture level of the granulation is between about 2.5% and about 4.5%. The residual moisture level of the granulation can also be between about 3% and about 3.75%, e.g., about 3.5%. In another aspect, the invention provides dried granulations comprising 5-methyl-2-(2′-chloro-6′-fluoroanilino)phenylacetic acid, croscarmellose sodium, and povidone, where the residual moisture level of the granulation is between about 1.5% and about 4%, e.g., between about 1.7% and about 3.5%, e.g., between about 2% and about 3%, e.g., about 2.5%. The aforementioned granulations are useful in making tablets that contain, e.g., 50, 100, 200, or 400 mg of 5-methyl-2-(2′-chloro-6′-fluoroanilino)phenylacetic acid, which tablets will have residual moisture levels corresponding to the level in the dried granulation used to make the tablet.

In another aspect, the invention provide methods for stabilizing 5-methyl-2-(2′-chloro-6′-fluoroanilino)phenylacetic acid in a pharmaceutical composition. The method comprises producing a solid pharmaceutical composition comprising 5-methyl-2-(2′-chloro-6′-fluoroanilino)phenylacetic acid, wherein the production yields a composition with a residual moisture level (“LOD”) between about 1.5% and about 5%. In certain embodiments, the method will yield a composition comprising between about 50 and about 200 mg of 5-methyl-2-(2′-chloro-6′-fluoroanilino)phenylacetic acid, which will have an LOD between about 2% and 5%, or between about 2.1% and about 4.5%. In other embodiments, the method will yield a composition comprising about 400 mg of 5-methyl-2-(2′-chloro-6′-fluoroanilino)phenylacetic acid that will have an LOD between about 1.5% and about 4%, or between about 1.7% and about 3.5%. In certain embodiments, the compositions produced are tablets, and in other embodiments, film coated tablets.

The formulations useful in the practice of the invention are intended for oral use and may be prepared according to any method known to the art for the manufacture of solid pharmaceutical compositions. Such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

5-methyl-2-(2′-chloro-6′-fluoroanilino)phenylacetic acid has surprisingly been found to undergo a variety of degradation processes when formulated as solid dosage forms, e.g., tablets. Tablets with between about 50 and about 200 mg of active agent preferably have an LOD of 3.5% with a desirable range between about 2.1% and about 4.5%. 65% drug-loaded tablets with about 400 mg of active agent preferably have an LOD of about 2.5%, with a desirable range between about 1.7% and about 3.5%. It has unexpectedly been found that if the LOD in the tablets are kept within the aforementioned parameters, the active agent, i.e., 5-methyl-2-(2′-chloro-6-fluoro-anilino)phenylacetic acid, is more chemically stable.

It has been surprisingly found that the ranges set forth above are the optimum LOD window between two different pathways by which 5-methyl-2-(2′-chloro-6′-fluoroanilino)phenylacetic acid degrades, i.e., an oxidative pathway and a cyclic pathway. The compositions and methods of the invention provide solid pharmaceutical compositions for oral administration comprising 5-methyl-2-(2′-chloro-6′-fluoroanilino)phenylacetic acid with minimal levels of total degradation products

Oral dosage levels for 5-methyl-2-(2′-chloro-6′-fluoroanilino)phenylacetic acid can be on the order of between about 50 mg and about 1200 mg per patient per day. In a preferred embodiment, the effective amount is between about 50 and about 400 mg per day, e.g., 50 mg, 100 mg, 200 mg, 300 mg, or 400 mg per day. The amount of drug that may be combined with the carrier materials to produce a single dosage form will vary depending upon the size and weight of the recipient, the body composition of the recipient, and the particular mode of administration. For example, a formulation intended for oral administration by human recipients may typically contain between about 50 and about 400 mg of active agent compounded with an appropriate and convenient amount of carrier material which may vary from about 5 to about 95 percent of the total composition. However, dosage unit forms may contain drug in amounts of 50, 100, 200, 300, 400, 600, 800, or 1200 mg. In one embodiment, the pharmaceutical composition comprises between about 50 and about 1200 mg of the 5-methyl-2-(2′-chloro-6′-fluoroanilino)phenylacetic acid. In other embodiments, the pharmaceutical composition comprises about 50, 100, 200, 300, or 400 mg of 5-methyl-2-(2′-chloro-6′-fluoroanilino)phenylacetic acid. In a particular embodiment, the composition comprises a capsule or tablet. In another embodiment, the pharmaceutical composition comprises a film-coated tablet.

Typically, the compositions of the invention comprise 5-methyl-2-(2′-chloro-6′-fluoroanilino)phenylacetic acid at a drug loading level of 50% to 90% by weight based on the weight of the composition.

In a particular aspect, the invention provides a tablet comprising about 400 mg of 5-methyl-2-(2′-chloro-6′-fluoroanilino)phenylacetic acid, wherein the tablet comprises between about 60% and about 70% of 5-methyl-2-(2′-chloro-6′-fluoroanilino)phenylacetic acid by weight. The tablet may comprise about 65% of 5-methyl-2-(2′-chloro-6′-fluoroanilino)phenylacetic acid by weight. In another aspect, the invention provides a tablet comprising between about 50 and about 200 mg of 5-methyl-2-(2′-chloro-6′-fluoroanilino)phenylacetic acid, where the tablet comprises about 50% of 5-methyl-2-(2′-chloro-6′-fluoroanilino)phenylacetic acid by weight.

It will be understood, however, that the specific dose level for any particular patient will depend upon a variety of factors including the age, body weight, general health, sex, diet, time of administration, rate of excretion, drug combination and the type and severity of the particular disease undergoing therapy. For many patients, a once daily dosage range of between about 50 and about 1200 mg per day, or between about 100 and about 400 mg per day is indicated, e.g., 50, 100, 200, 300, or 400 mg per day.

The invention provides in a further aspect a highly compressed tablet with a high drug loading. The tablet may be small in dimension e.g. 10 to 20 mm in diameter, preferably 15 to 20 mm, most preferably 17 to 18 mm; 5 to 10 mm in width, preferably 6.5 to 7.5 mm. The thickness of the tablet is from 4 to 8 mm, preferably 4.5 to 6.5 mm, most preferably 5.8 mm. Compression forces of between 10 to 20 kilo Newtons are used to prepare the compressed tablet. Benefits of this high drug loading include improved bioavailability, release characteristics and compliance.

Following is a description by way of example only of compositions of the invention. In the tables, drug substance refers to 5-methyl-2-(2′-chloro-6′-fluoroanilino)phenylacetic acid

EXAMPLE 1
400 mg formulation composition

| Ingredient | Mg/dose |
|---|---|
| Drug substance | 400.00 |
| Croscarmellose sodium, NF | 13.20 |
| Povidone K30, USP | 40.60 |
| Purified water, USP | Qs |
| Microcrystalline Cellulose, NF (PH 102) | 144.90 |
| Croscarmellose sodium, NF | 13.20 |
| Magnesium Stearate, NF | 3.10 |
| Opadry, Global White 00F18296 | 15.20 |
| Opadry, Global Red 00F15613 | 2.50 |
| Opadry, Global Black 00F17713 | 0.30 |
| Purified Water, USP | Qs |
| Film Coated Tablet Weight | 633.00 |

EXAMPLE 2
400 mg formulation composition

| Ingredient | Mg/dose |
|---|---|
| Drug substance | 400.00 |
| Croscarmellose sodium, NF | 3.0 |
| Povidone K30, USP | 18.5 |
| Purified water, USP | Qs |
| Microcrystalline Cellulose, NF (PH 102) | 181.0 |
| Croscarmellose sodium, NF | 3.0 |
| Magnesium Stearate, NF | 9.5 |
| Opadry, Global White 00F18296 | 15.20 |
| Opadry, Global Red 00F15613 | 2.50 |
| Opadry, Global Black 00F17713 | 0.30 |
| Purified Water, USP | Qs |
| Film Coated Tablet Weight | 633.00 |

EXAMPLE 3
200 mg formulation composition

| Ingredient | Mg/dose |
|---|---|
| Drug substance | 200.0 |
| Lactose monohydrate, NF | 46.6 |
| Microcrystalline Cellulose, NF (PH 101) | 51.4 |
| Croscarmellose sodium, NF | 4.0 |
| Titanium dioxide, USP | 8.0 |
| Povidone K30, USP | 16.0 |
| Purified water, USP | Qs |
| Microcrystalline Cellulose, NF (PH 102) | 52.0 |
| Croscarmellose sodium, NF | 12.0 |
| Titanium dioxide, USP | 8.0 |
| Magnesium Stearate, NF | 2.0 |
| Opadry, Global White 00F18296 | 11.8244 |
| Opadry, Global Red 00F15613 | 1.9642 |
| Opadry, Global Black 00F17713 | 0.2114 |
| Purified Water, USP | Qs |
| Film Coated Tablet Weight | 414.0 |

EXAMPLE 4
200 mg formulation composition

| Ingredient | Mg/dose |
|---|---|
| Drug substance | 200.0 |
| Lactose monohydrate, NF | 60.0 |
| Microcrystalline Cellulose, NF (PH 101) | 64.0 |
| Croscarmellose sodium, NF | 3.5 |
| Titanium dioxide, USP | 2.0 |
| Povidone K30, USP | 10.0 |
| Purified water, USP | Qs |
| Microcrystalline Cellulose, NF (PH 102) | 39.0 |
| Croscarmellose sodium, NF | 3.5 |
| Titanium dioxide, USP | 12.0 |
| Magnesium Stearate, NF | 6.0 |
| Opadry, Global White 00F18296 | 11.8244 |
| Opadry, Global Red 00F15613 | 1.9642 |
| Opadry, Global Black 00F17713 | 0.2114 |
| Purified Water, USP | Qs |
| Film Coated Tablet Weight | 414.0 |

EXAMPLE 5
100 mg formulation composition

| Ingredient | Mg/dose |
|---|---|
| Drug substance | 100.0 |
| Lactose monohydrate, NF | 23.3 |
| Microcrystalline Cellulose, NF (PH 101) | 25.7 |
| Croscarmellose sodium, NF | 2.0 |
| Titanium dioxide, USP | 4.0 |
| Povidone K30, USP | 8.0 |
| Purified water, USP | Qs |
| Microcrystalline Cellulose, NF (PH 102) | 26.0 |
| Croscarmellose sodium, NF | 6.0 |
| Titanium dioxide, USP | 4.0 |
| Magnesium Stearate, NF | 1.0 |
| Opadry, Global White 00F18296 | 5.9122 |
| Opadry, Global Red 00F15613 | 0.9821 |
| Opadry, Global Black 00F17713 | 0.1057 |
| Purified Water, USP | Qs |
| Film Coated Tablet Weight | 207.0 |

EXAMPLE 6
100 mg formulation composition

| Ingredient | Mg/dose |
|---|---|
| Drug substance | 100.0 |
| Lactose monohydrate, NF | 30.0 |
| Microcrystalline Cellulose, NF (PH 101) | 32.0 |
| Croscarmellose sodium, NF | 1.75 |
| Titanium dioxide, USP | 1.0 |
| Povidone K30, USP | 5.0 |
| Purified water, USP | Qs |
| Microcrystalline Cellulose, NF (PH 102) | 19.5 |
| Croscarmellose sodium, NF | 1.75 |
| Titanium dioxide, USP | 6.0 |
| Magnesium Stearate, NF | 3.0 |
| Opadry, Global White 00F18296 | 5.9122 |
| Opadry, Global Red 00F15613 | 0.9821 |
| Opadry, Global Black 00F17713 | 0.1057 |
| Purified Water, USP | Qs |
| Film Coated Tablet Weight | 207.0 |

EXAMPLE 7
50 mg formulation composition

| Ingredient | Mg/dose |
|---|---|
| Drug substance | 50.0 |
| Lactose monohydrate, NF | 11.7 |
| Microcrystalline Cellulose, NF (PH 101) | 12.8 |
| Croscarmellose sodium, NF | 1.0 |
| Titanium dioxide, USP | 2.0 |
| Povidone K30, USP | 4.0 |
| Purified water, USP | Qs |
| Microcrystalline Cellulose, NF (PH 102) | 13.0 |
| Croscarmellose sodium, NF | 3.0 |
| Titanium dioxide, USP | 2.0 |
| Magnesium Stearate, NF | 0.5 |
| Opadry, Global White 00F18296 | 2.9561 |
| Opadry, Global Red 00F15613 | 0.4910 |
| Opadry, Global Black 00F17713 | 0.0528 |
| Purified Water, USP | Qs |
| Film Coated Tablet Weight | 103.5 |

EXAMPLE 8
50 mg formulation composition

| Ingredient | Mg/dose |
|---|---|
| Drug substance | 50.0 |
| Lactose monohydrate, NF | 15.0 |
| Microcrystalline Cellulose, NF (PH 101) | 16.0 |
| Croscarmellose sodium, NF | 0.875 |
| Titanium dioxide, USP | 0.5 |
| Povidone K30, USP | 2.5 |
| Purified water, USP | Qs |
| Microcrystalline Cellulose, NF (PH 102) | 9.75 |
| Croscarmellose sodium, NF | 0.875 |
| Titanium dioxide, USP | 3.0 |
| Magnesium Stearate, NF | 1.5 |
| Opadry, Global White 00F18296 | 2.9561 |
| Opadry, Global Red 00F15613 | 0.4910 |
| Opadry, Global Black 00F17713 | 0.0528 |
| Purified Water, USP | Qs |
| Film Coated Tablet Weight | 103.5 |

Tablets other than 400 mg tablets are made as follows. Povidone is dissolved in water. The drug substance, lactose, microcrystalline cellulose PH101, croscarmellose sodium, titanium dioxide are mixed in a high shear mixer for 5 mins. This mixture is granulated with povidone solution pumped at a rate of 3 Kg/min. The resulting wet mixture is further mixed for 90 seconds after all the solution is added. The wet granulation is dried in a fluid bed dryer, using an inlet air temperature of 50 C. The residual moisture is 3.5% (target range of 2.1-4.5). Tthe dried granulation is milled through an 18 mesh screen. Extragranular excipients such as microcrystalline cellulose PH102, croscarmellose sodium and titanium dioxide are passed through an 18 mesh screen and mixed with the milled dried granulation in a blender for 200 revolutions to form a penultimate mixture. Magnesium stearate is passed through a 20 mesh hand screen and is mixed with the penultimate mixture in the blender for 50 revolutions to form a tableting mixture. The tableting mixture is pressed into tablets using a tablet press and oval punches. The coating powders (Opadry) are mixed with purified water to make a 15% w/w coating suspension. The tablets are film coated with the coating suspension in a coating pan using 60° C. to 75° C. inlet air temperature.

400 mg tablets are made as follows. Povidone is dissolved in water. The drug substance and croscarmellose sodium are mixed in a high shear mixer for 5 mins. This mixture is granulated with povidone solution pumped at a rate of 5.5 Kg/min. The resulting wet mixture is further mixed for 300 seconds after all the solution is added. The wet granulation is dried in a fluid bed dryer, using an inlet air temperature of 50 C. The residual moisture is 2.5% (acceptable range of 1.7-3.5). The dried granulation is milled through an 18 mesh screen. Extragranular excipients such as microcrystalline cellulose PH102 and croscarmellose sodium are passed through an 18 mesh screen and mixed with the milled dried granulation in a blender for 200 revolutions to form a penultimate mixture. Magnesium stearate is passed through a 20 mesh hand screen and is mixed with the penultimate mixture in the blender for 50 revolutions to form a tableting mixture. The tableting mixture is pressed into tablets using a tablet press and oval punches. The coating powders (Opadry) are mixed with purified water to make a 15% w/w coating suspension. The tablets are film coated with the coating suspension in a coating pan using 60° C. to 75° C. inlet air temperature.

All patents, patent applications, and other publications referred to herein are hereby expressly incorporated by reference in their entirety. In case of a conflict between the present specification and material incorporated by reference, the present specification is controlling.

What we claim is:

1. A solid pharmaceutical composition for treating a cyclooxygenase-2 dependent disorder or condition comprising between about 50 and about 400 mg of 5-methyl-2-(2′-chloro-6′-fluoroanilino)phenylacetic acid, wherein the residual moisture levels of said composition is between about 1.5% and about 5%.

2. The solid pharmaceutical composition of claim 1 comprising between about 50 and about 200 mg of 5-methyl-2-(2′-chloro-6′-fluoroanilino)phenylacetic acid, wherein the residual moisture level of said composition is between about 2% and about 5%.

3. The solid pharmaceutical composition of claim 2 wherein the residual moisture level of said composition is between about 2.1% and about 4.5%.

4. The solid pharmaceutical composition of claim 3, wherein the residual moisture level of said composition is about 3.5%.

5. The solid pharmaceutical composition of claim 1 comprising about 400 mg of 5-methyl-2-(2′-chloro-6′-fluoroanilino)phenylacetic acid, wherein the residual moisture level of said composition is between about 1.5% and about 4%.

6. The solid pharmaceutical composition of claim 5 comprising about 400 mg of 5-methyl-2-(2′-chloro-6′-fluoroanilino)phenylacetic acid, wherein the residual moisture level of said composition is between about 1.7% and about 3.5%.

7. The solid pharmaceutical composition of claim 6, wherein the residual moisture level of said composition is about 2.5%.

8. The solid pharmaceutical composition of claim 3, wherein said composition is a tablet.

9. The solid pharmaceutical composition of claim 4, wherein said composition is a tablet.

10. The solid pharmaceutical composition of claim 6, wherein said composition is a tablet.

11. The solid pharmaceutical composition of claim 7, wherein said composition is a tablet.

12. A method for stabilizing 5-methyl-2-(2′-chloro-6′-fluoroanilino)phenylacetic acid in a solid pharmaceutical composition, comprising producing a solid pharmaceutical composition comprising 5-methyl-2-(2′-chloro-6′-fluoroanilino)phenylacetic acid wherein said composition has a residual moisture level between about 1.5% and about 5%.

13. The method of claim 12, wherein said solid pharmaceutical composition comprises between about 50 and about 200 mg of 5-methyl-2-(2′-chloro-6′-fluoroanilino)phenylacetic acid, and wherein the residual moisture level of said composition is between about 2% and about 5%.

14. The method of claim 13 wherein the residual moisture level of said composition is between about 2.1% and about 4.5%.

15. The method of claim 14 wherein the residual moisture level of said composition is between about 3% and about 4%.

16. The method of claim 15 wherein the residual moisture level of said composition is about 3.5%.

17. The method of claim 12, wherein said solid pharmaceutical composition comprises about 400 mg of 5-methyl-2-(2′-chloro-6′-fluoroanilino)phenylacetic acid, and wherein the residual moisture level of said composition is between about 1.5% and about 4%.

18. The method of claim 17 wherein the residual moisture level of said composition is between about 1.7% and about 3.5%.

19. The method of claim 18 wherein the residual moisture level of said composition is between about 2% and about 3%.

20. The method of claim 19 wherein the residual moisture level of said composition is about 2.5%.

21. A dried granulation comprising 5-methyl-2-(2′-chloro-6′-fluoroanilino)phenylacetic acid, microcrystalline cellulose, lactose monohydrate, croscarmellose sodium, wherein the residual moisture level of said granulation is between about 2.5% and about 4.5%.

22. The dried granulation of claim 21, wherein the residual moisture level of said granulation is between about 3% and about 3.75%.

23. The dried granulation of claim 22, wherein the residual moisture level of said granulation is about 3.5%.

24. A dried granulation comprising 5-methyl-2-(2′-chloro-6′-fluoroanilino)phenylacetic acid, croscarmellose sodium, povidone, wherein the residual moisture level of said granulation is between about 1.5% and about 4%.

25. The dried granulation of claim 24, wherein the residual moisture level of said granulation is between about 1.7% and about 3.5%.

26. The dried granulation of claim 25, wherein the residual moisture level of said granulation is between about 2% and about 3%.

27. The dried granulation of claim 26, wherein the residual moisture level of said granulation is about 2.5%.

28. A solid pharmaceutical composition comprising the dried granulation of claim 21.

29. A solid pharmaceutical composition comprising the dried granulation of claim 22.

30. A solid pharmaceutical composition comprising the dried granulation of claim 25.

31. A solid pharmaceutical composition comprising the dried granulation of claim 26.
